(19) United States
(12) Patent Application Publication
(10) Pub. No.: US 2023/0088535 A1
(43) Pub. Date: Mar. 23, 2023

(54) ANALYSING A SUBJECT'S GRIP

(71) Applicant: KONINKLIJKE PHILIPS N.V., EINDHOVEN (NL)

(72) Inventors: Daniel Jason Schulman, Cambridge, MA (US); Mladen Milosevic, Cambridge, MA (US)

(21) Appl. No.: 17/948,285

(22) Filed: Sep. 20, 2022

Related U.S. Application Data
(60) Provisional application No. 63/246,009, filed on Sep. 20, 2021.

(51) Int. Cl. A61B 5/11; G01D 5/24; G01L 1/20

(52) U.S. Cl. CPC A61B 5/1125; G01D 5/2405; G01L 1/20

(57) ABSTRACT

According to an aspect, there is provided a grip measurement system (300) comprising a door handle (100') comprising a first sensor (108) configured to measure a first parameter indicative of an amount of a part of a user’s hand in contact with the first sensor; a storage device (304); and a processor (302) configured to estimate, based on the measured first parameter, a force applied onto the first sensor by the part of the user’s hand; and store an indication of the estimated force in the storage device.

### 1. FIELD OF THE INVENTION

The invention relates to analysing a subject’s grip and, more particularly, to analysing the subject’s hand grip based on the way the user interacts with a door handle.

### BACKGROUND OF THE INVENTION

Hand function, such as grip, is a key determinant of the ability to perform daily activities for a person suffering from one or more conditions, including rheumatoid arthritis and a variety of neurological and neuromuscular disabilities. Consequently, assessment of hand function can be clinically useful in several contexts, including in evaluating disease progress, developing and evaluating care plans, as an indicator of overall frailty, and as a predictor of mortality, hospitalization, and future physical functioning or disability. Assessment of hand function may also be useful in identifying risks to safety and/or identifying the ability to maintain independence in a person’s home. Grip strength assessment may be useful in clinical decisions for several common scenarios, including management of common physical disabilities (e.g., rheumatoid arthritis), physical and/or cognitive rehabilitation (e.g., stroke rehabilitation), and management of common conditions such as cardiovascular conditions.

Clinical assessment of hand function may be performed using one or more methods including a patient self-reporting physical function, pain, and their ability to perform daily tasks (e.g., using the Duruöz Hand Index or the Michigan Hand Outcome Questionnaire); grip strength and pinch strength, typically measured by a dynamometer; measurement of dexterity (e.g., finger flexion); and performance of a standardized set of tasks requiring hand function (e.g., the Arthritis Hand Function Test).

A full hand function assessment requires a combination of self-reporting and specialized equipment, such as a Jamar dynamometer, which measures maximal grip strength using a hydraulic device, and which is relatively expensive. It would be preferable to be able to accurately assess a person’s grip using a simplified measurement mechanism, as performing multiple assessment tasks may be tiring and painful for the target patient population. Moreover, accurate and thorough assessment requires clinician training and careful adherence to standardized protocols.

Some efforts to develop hand function assessment that can be used in a person’s home, potentially by unsupervised patients, have focused on assessment of grip strength, for example using a “grip ball” device. Other examples use sensors (e.g., via a “sensor glove”) to assess hand function.

Use of specialist equipment can be difficult for some people, particularly the elderly, for example. Moreover, a person might forget or be unable to use the equipment regularly, so a meaningful assessment might not be possible. Thus, there is a desire for a system that can be used to assess a person’s grip without putting undue pressure on the person, and which can be used without a clinician’s guidance or supervision.

### SUMMARY OF THE INVENTION

Measuring and/or analysing a person’s grip or hand function can be difficult, time-consuming and expensive using existing methods. The inventors of the present disclosure have recognised that there is a need for an improved system for analysing a person’s grip. Embodiments disclosed herein provide a grip measurement system that can be used to analyse a person’s grip in an unobtrusive manner, without the presence of a trained clinician, and without the person having to use a separate, complicated grip measurement device. This is achieved in the present invention through the use of sensors embedded in a door handle. The sensors are capable of measuring a parameter relating to the person’s hand function when the person uses the door handle, for example to open and/or close a door. In this way, the assessment can be made automatically, whenever the person operates the door handle, without impacting on the person’s everyday life.

According to a first specific aspect, there is provided a grip measurement system comprising: a door handle comprising a first sensor configured to measure a first parameter indicative of an amount of a part of a user’s hand in contact with the first sensor; a storage device; and a processor configured to estimate, based on the measured first parameter, a force applied onto the first sensor by the part of the user’s hand; and store an indication of the estimated force in the storage device.

In some embodiments, the processor may be configured to determine, based on the stored indication of the estimated force and an indication of one or more estimated forces stored previously in the storage device in respect of the part of the user’s hand, a change in the estimated force applied by the part of the user’s hand on the first sensor over a period of time.

The processor may be configured, responsive to determining that the change in the estimated force over the period of time meets a defined threshold condition, to generate an instruction signal for delivery to a recipient device.

In some embodiments, the processor may be configured to determine, based on the estimated force, an indication of a quality of the user’s grip on the door handle.

The grip assessment system may further comprise a user interface. The processor may be configured to provide an indication of the estimated force for presentation to a recipient via the user interface.

In some embodiments, the grip assessment system may further comprise a door sensor associated with a door on which the door handle is installed, the door sensor configured to acquire a measurement indicative of a force applied to the door by the user. The processor may be configured to estimate, based on the estimated force and the acquired measurement, an indication of the user’s strength applied when opening or closing the door.

In some embodiments, the part of the user’s hand may comprise a first finger. The door handle may comprise a second sensor configured to measure a second parameter indicative of an amount of a second finger of the user’s hand in contact with the second sensor. The first sensor may be positioned relative to the door handle so as to be contacted by the first finger of the user’s hand. The second sensor may be positioned relative to the door handle so as to be contacted by the second finger of the user’s hand. The processor may be configured to estimate, based on the measured second parameter, a force applied on the second sensor by the second finger of the user’s hand. The processor may be further configured to store an indication of the estimated force applied on the second sensor in the storage device.

The first sensor may comprise at least one of: a capacitive sensor; and a force sensitive resistor sensor.

According to a second aspect, there is provided a door handle comprising a first sensor configured to measure a parameter indicative of an amount of a part of a user’s hand in contact with the first sensor; and a processor configured to receive an indication of the parameter measured by the first sensor; estimate, based on the measured parameter, a force applied on the first sensor by the part of the user’s hand on the first sensor; and provide an indication of the estimated force for storage in a storage device.

In some embodiments, the door handle may further comprise a communication unit. The processor may be configured to transmit the indication of the estimated force for storage in a storage device using the communication unit.

According to third aspect, there is provided a computer-implemented method for analysing a person’s grip, the method comprising receiving, from a first sensor associated with a door handle, a measurement indicative of an amount of a part of a user’s hand in contact with the first sensor; estimating, based on the measurement, a force applied on the first sensor by the part of the user’s hand; and providing an indication of the estimated force for storage in a storage device.

The step of estimating the force applied on the first sensor may comprise estimating the force applied by the part of the user’s hand based on the surface area of the part of the user’s hand in contact with the first sensor.

In some embodiments, the method may further comprise determining, based on the estimated force and on an indication of one or more estimated forces measured previously in respect of the part of the user’s hand, a change in the estimated force applied by the part of the user’s hand on the first sensor over a period of time.

The method may further comprise providing at least one of: an indication of the estimated force; and an indication of the determined change in the estimated force for presentation to a recipient.

According to fourth aspect, there is provided a computer program product comprising a non-transitory computer-readable medium, the computer-readable medium having computer-readable code embodied therein, the computer-readable code being configured such that, on execution by a suitable computer or processor, the computer or processor is caused to perform steps of the methods disclosed herein.

These and other aspects will be apparent from and elucidated with reference to the embodiment(s) described hereinafter.

### BRIEF DESCRIPTION OF THE DRAWINGS

Exemplary embodiments will now be described, by way of example only, with reference to the following drawings, in which:

FIG. 1 is a schematic illustration of an example of a door handle according to various embodiments;

FIGS. 2A and 2B are schematic illustrations showing a finger and part of a door handle;

FIG. 3 is a schematic illustration of an example of a grip measurement system incorporating a door handle;

FIGS. 4A, 4B, 4C and 4D are schematic illustrations of examples of door handles according to various embodiments;

FIG. 5 is a schematic illustration of an example of a grip measurement system incorporated into a door;

FIG. 6 is a flowchart of an example of a method for analysing a person’s grip; and

FIG. 7 is a schematic illustration of an example of a processor in communication with a computer-readable medium.

### DETAILED DESCRIPTION OF EMBODIMENTS

Various embodiments disclosed herein provide a mechanism by which a person’s hand function (e.g., grip) can be measured and assessed using a device built into or forming part of a door handle. In this way, the user of the door handle (e.g., a person who is grip is to be assessed, such as a patient) may operate the door handle as part of their everyday life, in their own home, and measurements relating to their grip can be taken whenever the door handle is used. Thus, measurements can be taken in an unobtrusive manner, without the need for the user to use a separate piece of equipment. Moreover, as will become apparent below, the sensors incorporated into the door handle need not be complex sensors capable of accurately measuring a force applied onto the door handle, but may be less complex, lower cost sensors capable of measuring a parameter from which the applied force or pressure may be estimated.

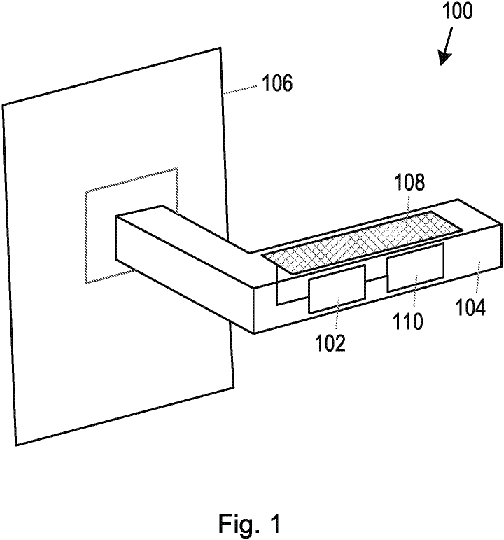

Referring now to the drawings, FIG. 1 is a schematic illustration of an example of a grip measurement device which is in the form of a door handle or door handle mechanism 100. A first aspect of the present invention relates to such a door handle. As will be apparent from the discussion below, and as will be understood to those skilled in the art, door handles may take various forms, and the door handle 100 shown in FIG. 1 is merely one example of a large number of possible door handles that could embody the grip measurement device or grip measurement system disclosed herein.

The door handle 100 comprises a processor 102 and a handle portion 104 which is configured to be engaged by the hand of a user during operation of the door handle. The handle portion 104 may be coupled to a door by a rotatable spindle (not shown in FIG. 1) that extends into the door through a panel 106, sometimes referred to as an escutcheon. As will be familiar to those skilled in the art, operation of the handle portion 104, by pushing down on the handle potion, may cause a latch to be retracted, enabling the door to be pushed or pulled from a closed position into an open position.

The door handle 100 comprises a first sensor 108 configured to measure a parameter indicative of an amount of a part of a user’s hand in contact with the first sensor. The first sensor 108 may, for example, be located on a surface of the handle portion 104 of the door handle 100, such as a top surface as shown in FIG. 1, in such a position that a user is able to touch the first sensor while operating the door handle. In some embodiments, a user may contact the first sensor 108 using a part or parts of their hand, such as their palm, their thumb, one or more of their fingers or a combination of these. For example, a user of the door handle 100 shown in FIG. 1 may place their palm or fingers of their right hand onto the handle portion 104 of the door handle 100, such that their fingers engage the first sensor 108. The user’s thumb may wrap around a bottom portion of the handle portion 104 to enable the user to grip the door handle 100. Operation of the door handle 100 may involve the user applying a downward pressure onto the first sensor 108. Some examples of the door handle 100 may include multiple sensors, for example an array of sensors (e.g., of a single type of a combination of types of sensors) arranged in a grid.

Rather than measuring a force or pressure applied by a part of the user’s hand, the first sensor 108 is adapted to measure a parameter indicative of an amount of the part of the user’s hand that is in contact with the first sensor. Thus, the first sensor 108 may comprise any sensor capable of measuring a surface area of an object touching it. When a part of a person’s hand applies a force onto a surface (e.g., a surface of a door handle), the surface area of the part of the user’s hand making contact with the surface increases as the force applied by the user is increased.

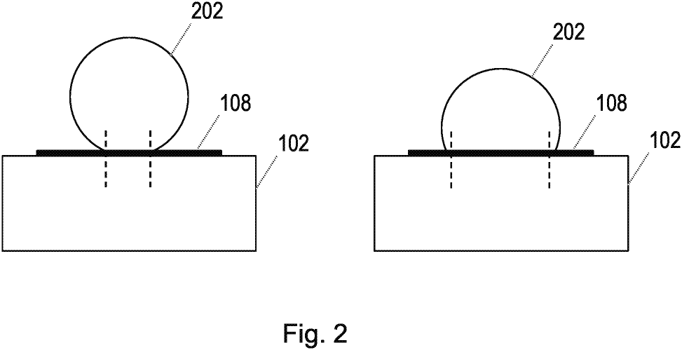

FIG. 2 shows schematically part of a door handle, such as the handle portion 104 of the door handle 100, and a portion of the user’s hand - in this case a finger 202 - in contact with the first sensor 108 of the door handle, with two different forces being applied. In FIG. 2A, the finger 202 is applying a relatively small force onto the first sensor 108, resulting in a relatively small surface area of the finger being in contact with the first sensor. In contrast, in FIG. 2B, the finger 202 is applying a relatively large force onto the first sensor 108, resulting in a relatively large surface area of the finger being in contact with the first sensor. According to embodiments disclosed herein, a force being applied onto the first sensor 108 by the finger 202 or by another part of the user’s hand can be estimated based on the surface area of the first sensor that is in contact with the user’s hand. While such an estimation may not provide an accurate measure of the force being applied, the estimated force is accurate enough for use in determining changes in the force is applied over time, and differences between the amount of force applied by different parts of the user’s hand. It will be appreciated that, rather than estimating a force applied onto the door handle 100, a pressure may alternatively be estimated.

In some embodiments, the first sensor 108 may comprise a capacitive sensor. Capacitive sensors, which will be familiar to those skilled in the art, include layers of material, between which is formed a uniform electrostatic field. A change in a capacitance measured at different positions of one of the layers can be used to detect the presence and position of part of the user’s hand that has come into contact with the sensor. In other embodiments, the first sensor 108 may comprise a force sensitive resistor type sensor. Force sensitive resistors, which will also be familiar to those skilled in the art, act as resistors that change their electrical resistance depending on how much they are pressed. Therefore, if a greater surface area touches a layer of material comprising multiple force sensitive resistors, then more of the resistors will change their resistor values, and the surface area in contact with the part of the user’s hand can be determined. In some examples, the first sensor 108 may comprise a combination of capacitive sensors and force sensitive resistors and, in other examples, other types of sensors may be used.

Referring again to FIG. 1, the processor 102 may, in some embodiments, be positioned within the door handle 100, for example within the handle portion 104 or within the escutcheon 106. In other embodiments, the processor 102 may be located elsewhere, such as within the door on which the door handle 100 is mounted, or remote from the door and door handle, and configured to communicate with other components of the door handle using wireless communication protocols. The processor 102 may, in some embodiments, be configured to communicate with multiple door handles and/or with other sensor components. The processor 102 is in communication with the first sensor 108 (e.g., via a wired or wireless connection), and is configured to perform various functions, such as steps of the methods disclosed herein. In some embodiments, the processor 102 is configured to receive an indication of the parameter measured by the first sensor 108. For example, the processor 102 may receive an indication of an amount (e.g., a surface area) that a part of a user’s hand (e.g., a finger) is in contact with the first sensor 108. The indication received by the processor 102 may, for example, comprise an indication of a number of points (e.g., sensing positions) on the first sensor that are in contact with the part of the user’s hand.

The processor 102 is configured to estimate, based on the measured parameter, a force applied on the first sensor 108 by the part of the user’s hand on the first sensor. In one example, the force applied on the first sensor 108 may be approximately proportional to the surface area of the first sensor that is in contact with the portion of the user’s hand (e.g., finger). The first sensor 108 may provide an output in the form of a voltage. A ratio of output voltage to the input voltage (Vout/Vin) is a measure of force; as the force applied increases, the resistance decreases, the output voltage approaches the input voltage, and this ratio approaches 1. It is also possible to convert the output of the first sensor 108 to other units, for example using a calibration curve. In other examples, the estimation of the force applied on the first sensor 108 may be more complex, as the surface area in contact with the first sensor may reach a maximum even though the force being applied is still increasing. Thus, in some examples, the processor 102 may be configured to determine when the surface area of the part of the user in contact with the first sensor 108 has stopped increasing or when the rate of increase of the surface area in contact with the first sensor is less than a defined threshold rate, and the force applied to the first sensor may be estimated at that threshold.

Estimating the force applied based on the surface area measured using the first sensor 108 may, in some embodiments, involve performing a calibration, to determine a surface area of one or more parts of the user (e.g., the user’s fingers, thumb, palm, and so on) when placed gently onto the door handle 100. In some examples, the user may be asked to gradually increase an amount of force applied to the door handle 100 during calibration, so that a determination can be made as to a minimum and maximum surface area likely to be registered by the first sensor 108. In some examples, the first sensor 108 may be configured to report a relative force rather than an absolute force, such that an improvement or decline in hand function over time can be determined.

The processor 102 is configured to provide an indication of the estimated force for storage in a storage device. In some embodiments, the storage device, which may comprise a memory unit, may be in wired or wireless communication with the processor 102, and may be located within the door handle 100 or remote from the door handle. For example, the storage device may comprise cloud-based storage.

By incorporating the first sensor 108 and the processor 102 within a door handle 100, estimates of the force applied by the user (e.g., by the user’s finger or hand) can be made while the user is performing a simple, everyday task (i.e., opening or closing a door), that does not require significant additional effort on the part of the user. Moreover, the first sensor 108 can be a relatively simple component capable of detecting a touch event when a user places their finger or hand on the handle portion 104 of the door handle 100. As such, relatively inexpensive sensing components may be used, rather than more expensive, complex sensors capable of accurately measuring a force applied onto a surface. As noted above, accurate force measurements are not required in scenarios involving the present invention, since relevant clinical information regarding the user can be determined using estimations of the force applied over time (e.g., on multiple occasions).

In embodiments in which the storage device is located in the door handle 100, and/or in which the processor 102 is in wired communication with the storage device, the processor may communicate directly with the storage device, for example to provide the indication of the estimated force storage. However, in embodiments in which the storage device is located remotely with respect to the door handle 100, the door handle may further comprise a communication unit 110 capable of transmitting/sending and/or receiving data. The processor 102 may be configured to transmit the indication of the estimated force for storage in a storage device using the communication unit 110. For example, if the storage device comprises cloud-based storage, then the communication unit 110 may upload the estimated force to the storage device via network (e.g., the Internet). The communication unit 110 may enable wired or wireless communication (e.g., using Bluetooth Low Energy (BLE), Z-wave, or the like).

In the example shown in FIG. 1, the door handle 100 includes both the first sensor 108 and the processor 102. Such a door handle 100 may be referred to as a “smart handle”. In other embodiments of the present disclosure, a door handle may include the first sensor 108, but other components may be remotely located with respect to the door handle. In other words, components may form part of a distributed system rather than part of a single device.

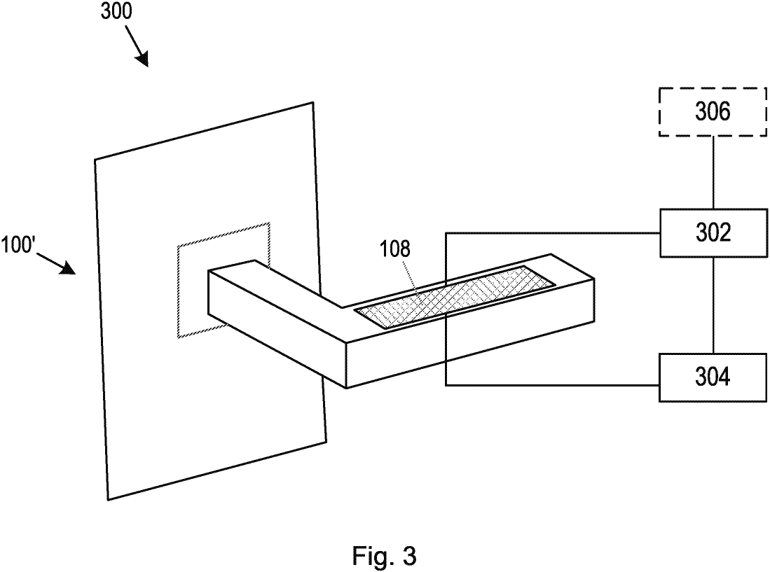

According to a further aspect, a system is provided. FIG. 3 is a schematic illustration of an example of a system 300, such as a system for measuring hand function, or a grip measurement system. The system 300 comprises a door handle 100', which may be similar to the door handle 100 shown in FIG. 1. The door handle 100' comprises a first sensor (e.g., the first sensor 108 discussed above) configured to measure a first parameter indicative of an amount of a part of a user’s hand in contact with the first sensor. The system 300 further comprises a processor 302 and a storage device 304. The processor 302 and the storage device 304 may be communication with one another and/or with the first sensor 108 of the door handle 100' via a wired or wireless connection.

The processor 302 is configured to estimate, based on the measured first parameter, a force applied onto the first sensor 108 by the part of the user’s hand. The processor 302 is further configured to store an indication of the estimated force in the storage device 304.

In some embodiments, the processor 102, 302 may be configured to determine, based on the stored indication of the estimated force and an indication of one or more estimated forces stored previously in the storage device 304 in respect of the part of the user’s hand, a change in the estimated force applied by the part of the user’s hand on the first sensor 108 over a period of time. The first sensor 108 may record measurements each time the user operates the door handle 100, 100' to open a door to which the door handle is attached. Each measurement may be recorded in the storage device 304, and may be associated with a time at which the measurement was recorded. Thus, in some embodiments, the system 300 may be considered to be a system for determining a change in grip ability of a person over a period of time.

In some embodiments, the measurements recorded using the first sensor 108 may be stored with data indicating an identity of the user. For example, a further sensor may detect the identity of the person using the door handle (e.g., through biometric sensing) or the user may identify themselves, for example using an RFID tag or the like. In other embodiments, the identity of the user may be determined or assumed based on the measured force.

Once at least two measurements are stored in the storage device 304, the processor 102, 302 is able to calculate a change in the force (i.e., the estimated force) applied by the part of the user’s hand (e.g., a finger) over the time period between which the at least two measurements were recorded. For example, if the user used the door handle 100, 100' to open a door at around 8 AM one morning and then again at around 8 AM the following morning, then the processor 102, 302 could calculate the change in the estimated force applied by the user on the door handle from one day to the next (e.g., over a period of 24 hours). With more measurements recorded and stored in the storage device 304, the processor 102, 302 is able to determine changes more regularly and/or over a longer period of time, and it is possible to detect trends in the measurements and, therefore, in the force applied by the part of the user’s hand. In this way, insights can be determined from the way in which the force changes over time. For example, if a force applied by one of the user’s fingers over a period of time is seen to steadily decrease, then it may be determined that the user’s strength in that finger is deteriorating, and treatment may be required to improve their strength. Similarly, if the user has suffered an injury to a finger, the force applied by their finger may be measured over time and, if the force is seen to increase steadily, then it may be determined that the rehabilitation of that finger has been successful.

In some embodiments, the processor 102, 302 may be configured, responsive to determining that the change in the estimated force over the period of time meets a defined threshold condition, to generate an instruction signal for delivery to a recipient device. For example, if the change in the estimated force decreases steadily over a period of time, it may be determined that the strength in the part of the user’s hand (e.g., a finger) is deteriorating. If the decrease in the estimated force falls below a threshold force, or if it is determined that the force applied over time decreases quicker than a defined threshold rate, then this could be indicative of a potentially serious medical issue in the user’s hand, and warning or alert may be generated. Thus, the instruction signal generated by the processor 102, 302 may comprise an instruction to cause the generation of an alert signal by a medical professional’s device (e.g., smart phone or computer). Appropriate action may then be taken to prevent further deterioration and/or to investigate potential causes of the deterioration in strength.

The processor 102, 302 may, in some embodiments, be configured to determine, based on the estimated force, an indication of a quality of the user’s grip on the door handle. For example, the first sensor 108 may be sized and positioned such that all four fingers of a user’s hand may come into contact with the first sensor when the user operates the door handle 100, 100'. The force estimated based on the contact area between the users fingers and the first sensor 108 increases as the user’s grip on the door handle 100, 100' is tightened. Therefore, if the applied force is estimated to be relatively low, then it may be determined that the user’s grip on the door handle 100, 100' is not very strong (i.e., poor quality) while, if the applied force is estimated to be relatively high, then it may be determined that the user’s grip on the door handle is relatively strong (i.e., good quality). The quality of the user’s grip may be indicative of the strength of the user’s hand, and knowledge of this may assist a medical professional in determining whether any rehabilitative action is required to improve (e.g., increase) the strength in the user’s hand.

Outputs from the processor may be provided to a recipient via any suitable user interface. In some embodiments, the system 300 may further comprise a user interface 306. The user interface 306 is optional, as indicated by the use of dashed lines in FIG. 3. The user interface 306 may, in some embodiments, be located remotely with respect to the door handle 100, 100' while, in other embodiments, the user interface may be integrated into the door handle, for example in the form of a display visible to the user. The processor 102, 302 may be configured to provide an indication of the estimated force for presentation to a recipient via the user interface 306. The recipient of the indication may be the user, a therapist, a medical professional, or the like.

In embodiments discussed so far, the door handle 100, 100' has included just one sensor, namely the first sensor 108. In other embodiments, however, the door handle 100, 100' may include multiple sensors, such that measurements may be taken at multiple positions on the handle portion 104 of the door handle 100, 100'.

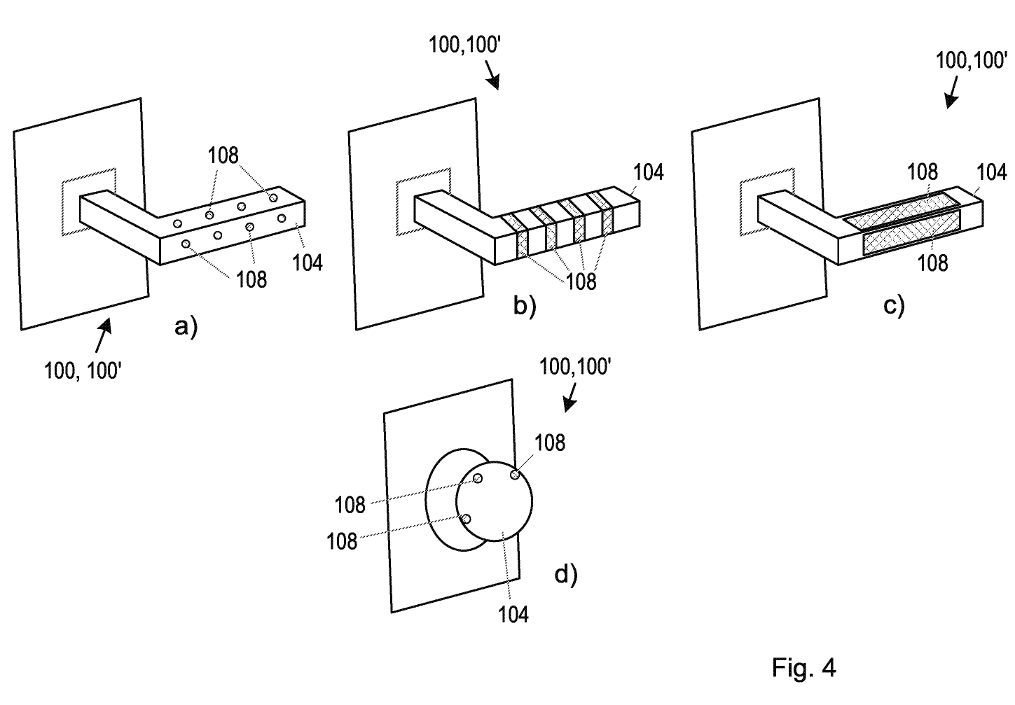

FIG. 4 illustrates 4 different examples of door handles having been different arrangements of sensors. The examples shown in FIGS. 4A, 4B and 4C are lever-type door handles 100, 100', while the example shown in FIG. 4D is a doorknob. As used herein, the term “door handle” is intended to cover both door handles of the type shown in FIGS. 4A, 4B and 4C and doorknobs of the type shown in FIG. 4D, and other types of devices or mechanisms used for opening doors. Similarly, in addition to handles used for opening swing-type doors that pivot on hinges, the term “door handle” as used herein is intended to cover handles used to open and close other types of doors, such as sliding doors, rotating doors, up-and-over doors, and the like. The door handle may be located on a door, or on another unit, such as a cabinet, a cupboard, a refrigerator or another appliance.

In FIG. 4A, the door handle 100, 100' includes a plurality of discrete sensors 108 located along the length of the handle portion 104 of the door handle. The shape of the sensors 108 is merely illustrative, and the sensors may be any suitable shape. In the embodiment shown in FIG. 4A, the door handle 100, 100' includes 16 sensors 108, with four sensors positioned on each side of the handle portion 104. The sensors 108 may be spaced apart from one another in such a way that they are positioned relative to fingers of a user’s hand. In this way, each finger of the user’s hand may contact at least one sensor 108 on at least one side of the handle portion 104.

In FIG. 4B, the door handle 100, 100' includes a plurality of continuous sensor sections formed around the perimeter of the handle portion 104. These ring-like sensors 108 may also be positioned such that, when a user operates the door handle 100, 100' with their hand, each of the user’s fingers on the hand engages with a corresponding sensor 108.

In FIG. 4C, the door handle 100, 100' includes a single sensor on each side of the handle portion 104. Measurements from each of the sensors 108 may be combined to calculate an overall force applied by the user’s hand onto the door handle 100, 100' during operation. In some examples, the sensors 108 shown in the embodiment of FIG. 4C may not be capable of determining a force applied by each individual finger of the user’s hand, since multiple fingers are in contact with each sensor during use. In other examples, however, processing techniques may be used to distinguish between contact made on the sensors 108 by different fingers on the user’s hand. In a variation of the example shown in FIG. 4C, a single sensor 108 may be formed around the perimeter of the handle portion 104, similar to the sensors shown in FIG. 4B.

In FIG. 4D, the door handle 100, 100' is in the form of a round doorknob. During operation, a user grabs the doorknob 100, 100' and rotates it order to release the latch. In this example, multiple sensors 108 are located around the circumference of the handle portion 104 of the doorknob. As in the example shown in FIG. 4A, the sensors 108 may be spaced apart and positioned in such a way that each of the fingers of the user’s hand contacts one of the sensors as the user holds the doorknob.

In any of the examples, more or fewer sensors 108 may be provided and arranged in any suitable arrangement. The first sensor 108 and any other sensors may be provided on or in (e.g., embedded in) the door handle 100, 100'.

Thus, in some embodiments, the part of the user’s hand may comprise a first finger. The door handle 100, 100' may comprise a second sensor configured to measure a second parameter indicative of an amount of a second finger of the user’s hand in contact with the second sensor. In such examples, the first sensor 108 may be positioned relative to the door handle 100, 100' so as to be contacted by the first finger of the user’s hand. The second sensor may be positioned relative to the door handle so as to be contacted by the second finger of the user’s hand. The processor 102, 302 may be configured to estimate, based on the measured second parameter, a force applied on the second sensor by the second finger of the user’s hand. The force applied by the second finger may be estimated in a similar way to the estimation of the force applied by the first finger. The processor 102, 302 may be configured to store an indication of the estimated force applied on the second sensor in the storage device. In some embodiments, the estimated force may be associated with an indication of the finger of the user (e.g., first finger, a second finger, or the like) that applied to the force. In other embodiments, the door handle 100, 100' may comprise further sensors (e.g., third sensor, a fourth sensor, or the like), each sensor configured and/or positioned to measure a parameter indicative of an amount of a corresponding finger of the user’s hand or portion of the hand in contact with that sensor.

Determining forces applied by individual fingers of a user’s hand, and determining changes over time in the force is applied by user’s fingers can be helpful in differentiating disease states in conditions such as arthritis.

In some embodiments, the storage device 304 may be configured to store data relating to multiple devices (e.g., door handles) within a home or multiple devices from different doors or homes. The storage device 304 may form part of an electronic health record (EHR), and the processor 102, 302 may be configured to retrieve data from other systems, such as medical data from the EHR, to combine data from various sources in the storage device. The storage device (and other components of the door handle 100, 100') may be configured to interact and/or interoperate with an EHR via one or more standards, such as the Fast Healthcare Interoperability Resources (FHIR) standard.

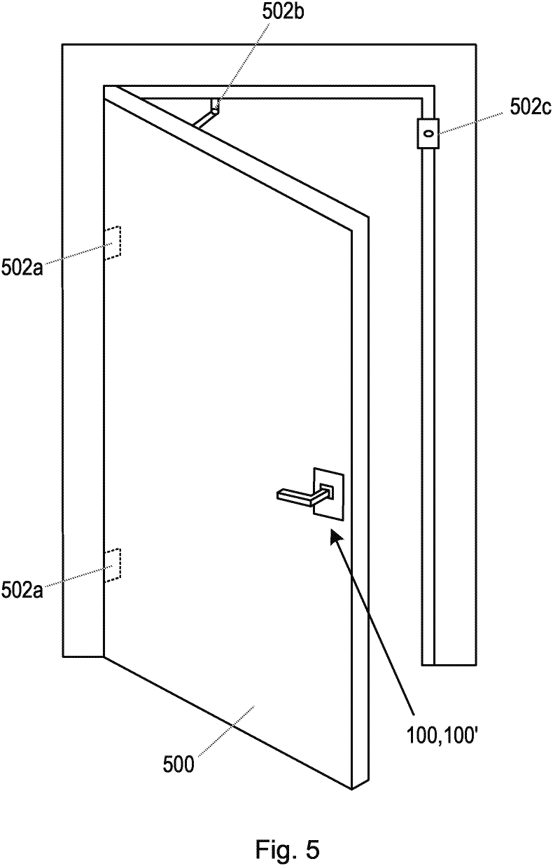

In some embodiments, measurements made using the first sensor 108 (and other sensors where available) may be combined with additional data in order to gain a more thorough understanding further insights regarding the strength and/or capabilities of the user’s hand or arm. FIG. 5 is an illustration of an example of a door 500 incorporating a door handle 100, 100' along with further sensors that can be used to measure additional data. In addition to the door handle 100, 100' and the first sensor 108, the door 500 may further include one or more additional sensors referred to as door sensors 502 configured to measure a force applied to the door by the user. The door sensor(s) 502 may be positioned at any suitable position relative to the door, such that a force applied onto the door can be measured.

In one example, a door sensor 502a may be located at a position corresponding to one or more hinges associated with the door 500. For example, a door sensor 502a may be positioned at, in or adjacent to a hinge, and configured to measure a force at which the door 500 is opened or closed. In another example, a door sensor 502b may be located in or on (or may form a part of) a closure mechanism of the door, or a mechanism intended to restrict the speed of a closing door. In another example, a door sensor 502c may be located on one or more of a frame, a casing, a jamb and a threshold associated with the door 500. Such a sensor may, for example, comprise an optical sensor (e.g., a laser sensing device) capable of detecting a speed at which the door 500 is closed or opened. In other examples, other types of sensors may be used.

Thus, the system 300 may further comprise a door sensor 502 associated with a door 500 on which the door handle 100, 100' is installed, the door sensor configured to acquire a measurement indicative of a force applied to the door by the user. The processor 102, 302 may be configured to estimate, based on the estimated force and the acquired measurement, an indication of the user’s strength applied when opening or closing the door 500. The measurements acquired using the door sensor 502 may, in one example, be combined with (e.g., added to) the estimated force in order to determine a combined indication of the user’s strength when opening or closing the door 500. If it is determined from the first sensor 108 that the user’s grip is relatively weak, but it is determined from the door sensor 502 that the strength applied when opening or closing the door 500 is relatively high, then it may be determined that the user’s grip strength should be improved. However, if it is determined that strength applied when opening or closing the door 500 is relatively low, but the user’s grip strength is relatively strong, then it may be determined that the user’s grip is adequate, but their arm strength should be improved.

In some embodiments, the processor 102, 302 may be configured to estimate and function, grip strength and/or a level of frailty of a user from data acquired during one or more grip events of the door handle 100, 100', and may further make use of other data, algorithms and/or rules, including rules created by experts in the relevant field. In some embodiments, machine learning models, for example decision trees and regression models, may be trained using historical and/or laboratory-gathered data relating to grip data (e.g., grip strength, force applied, and the like) combined with clinical assessment data. Such machine learning models may be used by the processor 102, 302 to estimate or determine a level of frailty based on data measured using the first sensor 108. The measured data may, in some examples, be supplemented by data provided by clinicians (e.g., via the user interface 306). For example, a clinician may provide details relating to the user, such as known disabilities, and this may be used by the processor 102, 302 to influence the hand function assessment. For example, a known disability may be taken into account, and forces estimated based on the measurements made using the first sensor 108 may be weighted accordingly.

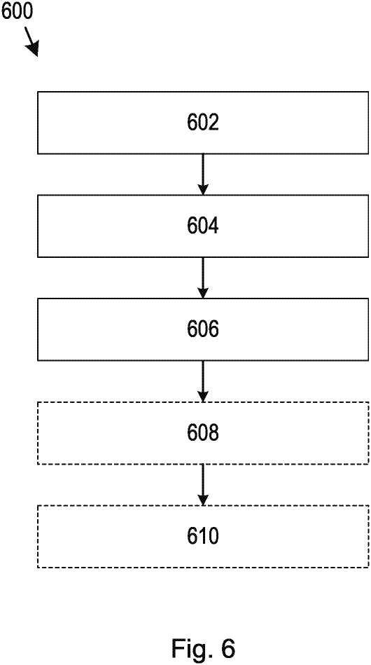

According to a further aspect of the invention, a method is provided. FIG. 6 is a flowchart of an example of a method 600. The method 600, which may comprise a method for analysing a person’s grip, may be a computer-implemented method, for example implemented using one or more processors, such as the processor 102, 302.

The method 600 comprises, at step 602, receiving, from a first sensor 108 associated with a door handle 100 100', a measurement indicative of an amount of a part of a user’s hand (e.g., a finger or a palm) in contact with the first sensor. The measurement may, for example, comprise a surface area of the first sensor 108 being contacted by the part of the user’s hand. At step 604, the method 600 comprises estimating, based on the measurement, a force applied on the first sensor 108 by the part of the user’s hand. The method 600 comprises, at step 606, providing an indication of the estimated force for storage in a storage device 304. For example, the indication may be provided by transmitting data representative of the estimated force, using a communication unit 110, to a storage device 304.

In some embodiments, the step of estimating the force applied on the first sensor 108 (i.e., step 604) may comprise estimating the force applied by the part of the user’s hand based on the surface area of the part of the user’s hand in contact with the first sensor.

The method 600 may, in some embodiments, further comprise one or more additional steps, for example steps corresponding to functions of the processor 102, 302 discussed herein. For example, the method 600 may further comprise, at step 608, determining, based on the estimated force and on an indication of one or more estimated forces measured previously in respect of the part of the user’s hand, a change in the estimated force applied by the part of the user’s hand on the first sensor over a period of time. By taking measurements in estimating force is applied at different locations over a period of time, it is possible to spot trends in the data, and this can lead to an early warning of a potential medical problem requiring treatment. For example, a downward trend in grip strength may be caused by an illness or injury leading to a decline in the user’s hand function. Likewise, an upward trend may indicate improvement, for example indicative of recovery from an injury.

At step 610, the method 600 may further comprise providing at least one of: an indication of the estimated force; and an indication of the determined change in the estimated force for presentation to a recipient. The recipient may be the user or a medical professional (e.g., a doctor) investigating the user’s hand function/grip strength. The data may be presented to the recipient using a display (e.g., a screen or touchscreen) of a computing device such as a desktop computer, a laptop computer, a tablet computer, a smart phone and/or a wearable device, or using some other device or presentation, such as a speaker.

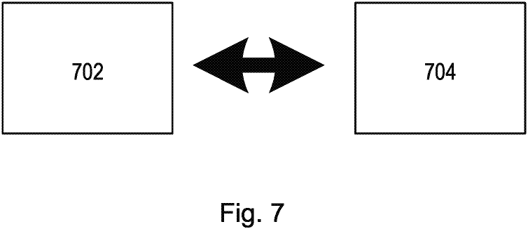

According to a further aspect, a computer program product is provided. FIG. 7 is a schematic illustration of an example of a processor 702 in communication with a computer readable medium 704. A computer program product comprises a non-transitory computer-readable medium 704, the computer-readable medium having computer-readable code embodied therein, the computer-readable code being configured such that, on execution by a suitable computer or processor 702, the computer or processor is caused to perform steps of the method 600 disclosed herein.

Embodiments of the present disclosure provide a mechanism by which a person’s grip can be analysed in an unobtrusive manner, using sensors attached to or embedded in a door handle, which may be used by the person repeatedly throughout the day. In this way, the person whose grip is to be analysed is not required to use any other specific grip measuring device, and can continue about his or her daily life, with the appropriate measurement being taken each time the person uses the door handle to open or close the door. The sensor is used to measure the data from which the force applied by the person is estimated can be relatively simple and inexpensive sensors meaning the cost of the overall device (e.g., the door handle) can be kept relatively low. Furthermore, simple sensors can be used in place of complex and accurate sensors because the exact force applied by the user’s hand is not required; rather, trends in the estimated force applied can be determined, and these can provide sufficient information regarding how the strength/grip ability/hand function of a user’s hand has changed over time. Even a single measurement/estimated force can be sufficient to identify significant issue relating to a user’s grip strength.

The processor 102, 302, 702 can comprise one or more processors, processing units, multi-core processors or modules that are configured or programmed to control components of the device (e.g., the door handle 100, 100') or system 300 in the manner described herein. In particular implementations, the processor 102, 302, 702 can comprise a plurality of software and/or hardware modules that are each configured to perform, or are for performing, individual or multiple steps of the method described herein.

The term “module”, as used herein is intended to include a hardware component, such as a processor or a component of a processor configured to perform a particular function, or a software component, such as a set of instruction data that has a particular function when executed by a processor.

It will be appreciated that the embodiments of the invention also apply to computer programs, particularly computer programs on or in a carrier, adapted to put the invention into practice. The program may be in the form of a source code, an object code, a code intermediate source and an object code such as in a partially compiled form, or in any other form suitable for use in the implementation of the method according to embodiments of the invention. It will also be appreciated that such a program may have many different architectural designs. For example, a program code implementing the functionality of the method or system according to the invention may be sub-divided into one or more sub-routines. Many different ways of distributing the functionality among these sub-routines will be apparent to the skilled person. The sub-routines may be stored together in one executable file to form a self-contained program. Such an executable file may comprise computer-executable instructions, for example, processor instructions and/or interpreter instructions (e.g., Java interpreter instructions). Alternatively, one or more or all of the sub-routines may be stored in at least one external library file and linked with a main program either statically or dynamically, e.g., at run-time. The main program contains at least one call to at least one of the sub-routines. The sub-routines may also comprise function calls to each other. An embodiment relating to a computer program product comprises computer-executable instructions corresponding to each processing stage of at least one of the methods set forth herein. These instructions may be sub-divided into sub-routines and/or stored in one or more files that may be linked statically or dynamically. Another embodiment relating to a computer program product comprises computer-executable instructions corresponding to each means of at least one of the systems and/or products set forth herein. These instructions may be sub-divided into sub-routines and/or stored in one or more files that may be linked statically or dynamically.

The carrier of a computer program may be any entity or device capable of carrying the program. For example, the carrier may include a data storage, such as a ROM, for example, a CD ROM or a semiconductor ROM, or a magnetic recording medium, for example, a hard disk. Furthermore, the carrier may be a transmissible carrier such as an electric or optical signal, which may be conveyed via electric or optical cable or by radio or other means. When the program is embodied in such a signal, the carrier may be constituted by such a cable or other device or means. Alternatively, the carrier may be an integrated circuit in which the program is embedded, the integrated circuit being adapted to perform, or used in the performance of, the relevant method.

Variations to the disclosed embodiments can be understood and effected by those skilled in the art in practicing the principles and techniques described herein, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. A computer program may be stored or distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

What is claimed is:

1. A grip measurement system comprising:
a door handle comprising:
a first sensor configured to measure a first parameter indicative of an amount of a part of a user’s hand in contact with the first sensor;
a storage device; and
a processor configured to: estimate, based on the measured first parameter, a force applied onto the first sensor by the part of the user’s hand; and store an indication of the estimated force in the storage device.

2. A grip assessment system according to claim 1, wherein the processor is configured to determine, based on the stored indication of the estimated force and an indication of one or more estimated forces stored previously in the storage device in respect of the part of the user’s hand, a change in the estimated force applied by the part of the user’s hand on the first sensor over a period of time.

3. A grip assessment system according to claim 2, wherein the processor is configured to, responsive to determining that the change in the estimated force over the period of time meets a defined threshold condition, generate an instruction signal for delivery to a recipient device.

4. A grip assessment system according to claim 1, wherein the processor is configured to determine, based on the estimated force, an indication of a quality of the user’s grip on the door handle.

5. A grip assessment system according to claim 1, further comprising a user interface, wherein the processor is configured to:
provide an indication of the estimated force for presentation to a recipient via the user interface.

6. A grip assessment system according to claim 1, further comprising a door sensor associated with a door on which the door handle is installed, the door sensor configured to acquire a measurement indicative of a force applied to the door by the user, wherein the processor is configured to estimate, based on the estimated force and the acquired measurement, an indication of the user’s strength applied when opening or closing the door.

7. A grip assessment system according to claim 1, wherein the part of the user’s hand comprises a first finger, and wherein the door handle comprises:
a second sensor configured to measure a second parameter indicative of an amount of a second finger of the user’s hand in contact with the second sensor;
wherein the first sensor is positioned relative to the door handle so as to be contacted by the first finger of the user’s hand;
wherein the second sensor is positioned relative to the door handle so as to be contacted by the second finger of the user’s hand;
wherein the processor configured to: estimate, based on the measured second parameter, a force applied on the second sensor by the second finger of the user’s hand; and
store an indication of the estimated force applied on the second sensor in the storage device.

8. A grip assessment system according to claim 1, wherein the first sensor comprises at least one of: a capacitive sensor; and a force sensitive resistor sensor.

9. A door handle comprising:
a first sensor configured to measure a parameter indicative of an amount of a part of a user’s hand in contact with the first sensor; and
a processor configured to:
receive an indication of the parameter measured by the first sensor; estimate, based on the measured parameter, a force applied on the first sensor by the part of the user’s hand on the first sensor; and
provide an indication of the estimated force for storage in a storage device.

10. A door handle according to claim 9, further comprising:
a communication unit, wherein the processor is configured to transmit the indication of the estimated force for storage in a storage device using the communication unit.

11. A computer-implemented method for analysing a person’s grip, the method comprising:
receiving, from a first sensor associated with a door handle, a measurement indicative of an amount of a part of a user’s hand in contact with the first sensor;
estimating, based on the measurement, a force applied on the first sensor by the part of the user’s hand; and
providing an indication of the estimated force for storage in a storage device.

12. A computer-implemented method according to claim 11, wherein estimating the force applied on the first sensor comprises estimating the force applied by the part of the user’s hand based on the surface area of the part of the user’s hand in contact with the first sensor.

13. A computer-implemented method according to claim 11, further comprising: determining, based on the estimated force and on an indication of one or more estimated forces measured previously in respect of the part of the user’s hand, a change in the estimated force applied by the part of the user’s hand on the first sensor over a period of time.

14. A computer-implemented method according to claim 13, further comprising: providing at least one of: an indication of the estimated force; and an indication of the determined change in the estimated force for presentation to a recipient.

15. A computer program product comprising a non-transitory computer-readable medium, the computer-readable medium having computer-readable code embodied therein, the computer-readable code being configured such that, on execution by a suitable computer or processor, the computer or processor is caused to perform the method of claim 11.
